(19) United States
(12) Patent Application Publication
(10) Pub. No.: US 2002/0035117 A1
(43) Pub. Date: Mar. 21, 2002

(54) Theophylline and 3-isobutyl-1-methylxanthine based N-7 substituted derivatives displaying inhibitory activities on type five phosphodiesterase

(76) Inventor: Ing-Jun Chen, Kaohsiung (TW)

Correspondence Address: BUCKMAN AND ARCHER, 600 OLD COUNTRY ROAD, GARDEN CITY, NY, 11530, US

(21) Appl. No.: 09/906,245

(22) Filed: Jul. 16, 2001

(30) Foreign Application Priority Data
Jul. 28, 2000 (TW) 89115304

(51) Int. Cl. A61K 31/522; C07D 473/04

(52) U.S. Cl. 514/263; 514/265; 544/267; 544/269

(57) ABSTRACT

Theophylline derivative of formula I and II,

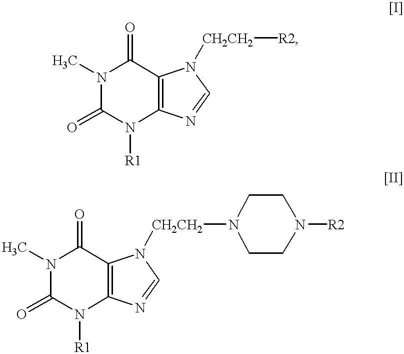

Wherein R₁ is —(CH₂),CH₃; R₂ is a member selected from the group of

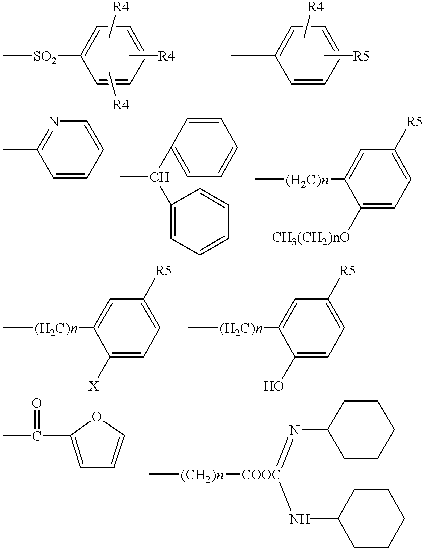

R₄ is a member selected from the group of H, —(CH₂)ₙCH₃, X, NH₂ and —NO₂;

R₅ is a member selected from the group of H,

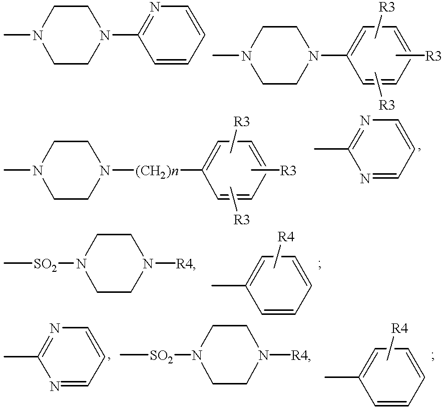

wherein R₃ is a member selected from the group of halogen, hydroxyl group (OH), saturated 1-3 straight chain carbon or group substituting one hydrogen; n is 0, 1, 2 or 3. In vivo or in vitro experiments, prove the carvernosal relaxation induced by these compounds.

### FIELD OF THE INVENTION

This invention relates to compounds of theophylline and 3-isobutyl-1-Miethylxanthine (IBMX) based on N-7 substituted derivatives, which upon laboratory testing on animals, have proven that they pharmacologically possesses inhibitory activities on type Five Phosphodiesterase, relaxation of corpus carvernosal smooth muscle, and increase of intracarvernosal pressure (ΔICP).

### BACKGROUND OF THE INVENTION

The endotheliuim plays a major role in regulating vascular smooth muscle (VSM) tone through the release of a variety of vasoactive factors. Among the endotheliuim-derived vasodilators, nitric oxide (NO) is probably the primary mediator of endotheliuim-dependent relaxation in most blood vessels. Nitric oxide in numerous bioregulatory pathways has not only expanded new therapeutic avenues for NO-related compounds but also led to an increased use of such compounds in pharmacological studies.

In recent years, nitric oxide has been shown to be an important regulator of vascular functions by controlling blood vessel tone as well as blood cell interactions with the vascular wall (S. Moncada et al., Pharmacol. Rev. vol. 43, No. 2, pp. 109-142, 1991). The action of NO (nitric oxide) as a vasodilator is mediated by the activation of vascular smooth muscle soluble guanylyl cyclase (sGC), a signal transduction enzyme that forms the second messenger molecular cyclic GMP (William P. Arnold et al., Proc. Natl. Acad. Sci. vol. 74, No. 8, pp. 3203-3207, 1977, Charles J. Lowenstein et al., Ann. Intern. Med. vol. 102, No. 3, pp. 227-237, 1994). The activity of several cyclic GMP (guanosine 3′, 5′-cyclic monophospliate) which lead to vasorelaxation has been determined. The membrane-bound guanylyl cyclases are receptor-like enzymes which are activated by extracellular binding of natriuretic peptides. In contrast, soluble guanylyl cyclases act via their haemoglobin group which is an important intracellular receptors for nitric oxide (Paulus Wohlfart et al., Br. J. Pharmcol. vol. 128, pp. 1316-1322, 1999). Moreover, the increases in cGMP with these guanylyl cyclase activators and phosphodiesterases (PDE) or cGMP breakdown inhibition with have been associated with the relaxation of vascular and tracheal smooth muscles.

The interactions between endogenous NO or NO donors and endothelium-derived hyperpolarizing factor (EDHF) or K⁺ channel activators have received a great deal of attention (Francisco Pérez-Vizcaino et al., Br. J. Pharmcol. vol. 123, pp. 847-854, 1998). K⁺ channels play a major role in the regulation of the resting membrane potential and modulate VSM (vascular smooth muscle) tone (Mark T. Nelson & John M. Quayle, Am. J. Physiol. vol. 268, C799˜C822, 1995). The endothelium-derived hyperpolarizing factor activates the potassium channels, and the potassium flux hyperpolarizes and thus relaxes the smooth muscle cell. Recent findings suggest that activation of endothelium K_{AT′P} channels (ATP-sensitive potassium channels) may also release endothelium-derived nitric oxide (Ethel C. Feleder & Edda Adler-Graschinsky, Eur. J. Pharmacol. vol. 319, pp. 229-238, 1997) or endothelium-derived hyperpolarizing factor (Richard White & C. Robin Hiley, Eur. J. Pharmacol. vol. 339, pp. 157-160, 1997). Nitric oxide donors have been shown to activate K_{ATP} channels via a cyclic GMP-dependent mechanism, presumably involving activation of cyclic GMP-dependent protein kinase, in rat aortic smooth muscle cells (Masaliiro Kubo et al., Circ. Res. vol. 74, No. 3, pp. 471-476, 1993) and rabbit mesenteric artery (Michael E. Murphy & Joseph E. Brayden, J. Physiol. vol. 486, No. 1, pp. 47-48, 1995), and by a cyclic GMP-independent mechanism in the rat miesenteric artery (Thomas Weidelt et al., J. Physiol. vol. 500, No. 3, pp. 617-630, 1997). Although most of the endothelium-dependent relaxation is due to NO (nitric oxide), hyperpolarization associated with K⁺ channels opening can supplement 60-80% of this response if NO synthesis is blocked is (E. V. Kilpatrick & T. M. Cocks, Br. J. Pharmacol. vol. 112, pp. 557-565, 1994).

The combination activity of soluble guanylyl cyclase (sGC) stimulation and K⁺ channels opening in a molecule, such as found in nicorandil, although shown without phosphodiesterase (PDE) inhibition activity, is able to relax agonist-induiced vasoconstriction more fully (F. Pérez-Vizcaino et al., Br. J. Pharmcol. vol. 123, pp. 847-854, 1998). YC-1(3-(5′-hydroxymetlhyl-2furyl)-1-benzyl-indazol) is a representative of a class of sGC activator with PDE (phosphodiesterase) inhibition and leads to a long-lasting cyclic GMP-mediated inhibition of vasoconstriction (Jan Galle et al., Br. J. Pharmcol. vol. 127, pp. 195-203, 1999).

### SUMMARY OF THE INVENTION

This invention covers compounds of theophylline and 3-isobutyl-1-methylxanthine (IBMX) base based N-7 substituted derivatives, which on laboratory testing or animals, have proven that they pharmacologically possesses Inhibitory activities on type Five Phosphodiesterase.

This invention also covers the synthetic methods of some novel theophylline derivatives.

### BRIEF DESCRIPTION OF THE TABLES AND FIGURES

The invention will now be described by way of example with reference to the accompanying Tables and Figures in which:

Table 1 physicochemical Data

Table 2 Rabbit Corpus Cavernosal Relaxation IC₅₀

Table 3 Rabbit Corpus Cavernosal Relaxation IC₅₀ on K⁺ channels blocker

Table 4 Show the increase of intracarvernous pressure (ΔICP) induced by the compounds.

FIG. 1 illustrates the structure of Nicorandil and glibenclaimde

FIG. 2 illustrates the synthetic method of the compounds of formula II

FIG. 3 illustrates the synthetic method for compounds of formula I

FIG. 4A-4B illustrates the experiment, effects of compound 14 on phenylephrine precontracted rabbit corpus cavernosal in the absence and presence of L-NAME, methylene blue, ODQ and potassium channel blockers. Significantly different from control, P<0.05 (two way repeated measures ANOVA followed by stydent-Newman-Keuls test)

FIG. 4A illustrates the experiment, effects of compound 14 on phenylephrine precontracted rabbit corpus cavernosal in the absence and presence of L-NAME, methylene blue, ODQ.

1. . . control group

2. . . L-NAME (100 μM)

3. . . methylene blue (10 μM)

4. . . ODQ (1 μM)

FIG. 4B illustrates the experiments, effects of compound 14 on phenylephrine precontracted rabbit corpus cavernosal in the absence and presence of potassium channel blockers.

5. . . . control group

6. . . . Glibenclamide (1 μM)

7. . . . TEA (10 mM)

8 . . . . 4-AP (100 μM)

FIG. 5 illustrates the additive effects, compound 14 and IBMX(3-Isobutyl-1-Methylxanthine) on phenylephrine precontracted rabbit carvernosal strips. Each value represent the mean·S. E., n=8.*P<0.05 as compared with the control value. (ANOVA followed by Dunnett's test).

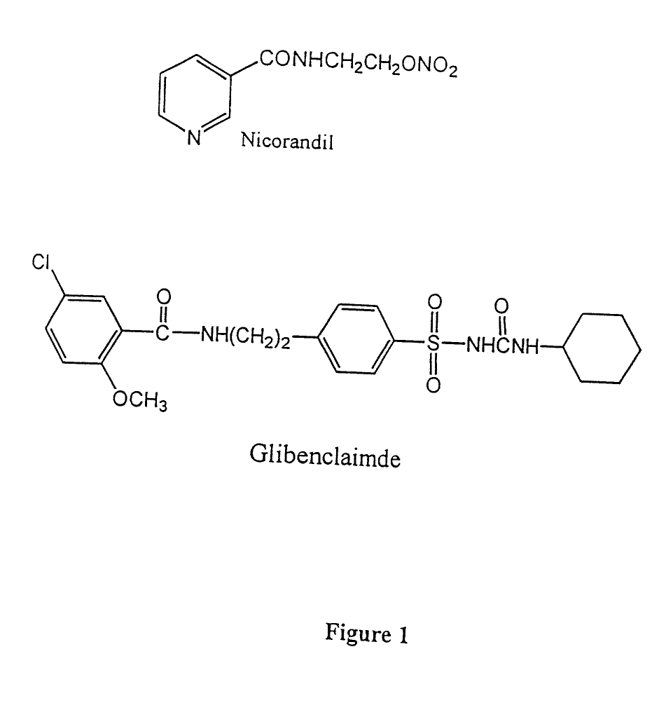

1. . . . control group

2. . . . IBMX

3. . . . compound 14

4. . . . compound 14 and IBMX

### DETAILED DESCRIPTION OF THE INVENTION

The invention covers theophylline derivative of formula I and II

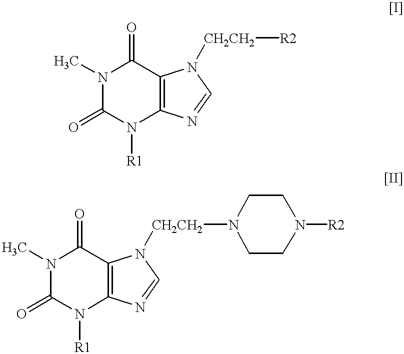

R₁ is present —(CH₂)ₙCH₃; R₂ is a member selected from the group consisting of

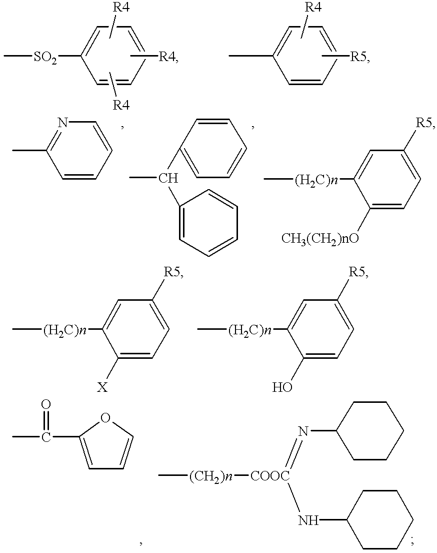

R₄ is a member selected from the group of H, —(CH₂)ᵤCH₃, X , —NH₂ and —NO₂;

R₅ is a member selected from the group consisting of H, and from the group consisting of

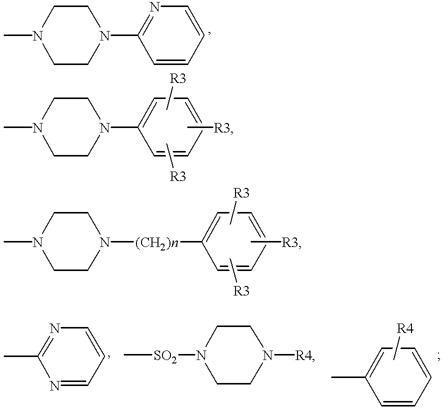

R₃ is a member select from the group consisting of halogen, hydroxyl group (OH), saturated 1-3 straight chain carbon atoms, or have substitute group replacing one hydrogen; the n, m are 0, 1, 2, or 3. When R₅ is selected as the group

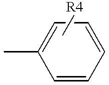

R₄ may be on the ortho, meta or the para position of the benzene ring. In vivo or in vitro experiments prove the carvernosal relaxation induced by these compound.

This invention covers compounds which involve a molecular modification based on theophylline and 3-isobutyl-1-methylxanthine (IBMX) and particularly compounds which are substituted derivatives on theophylline. In vivo test have proved that the compounds have the sGC (soluble guanylyl cyclase) stimulation, K⁺ channels opening and as a VSM(vascular smooth muscle) relaxant.

Recently it has been reported that theophylline has adenosine acceptor antagonist and phosphodiesterase (PDE) inhibitor function no matter whether positive 1 or 7 molecule modification based on methylxanthine is involved (Ken-Ichi Miyamoto et al., J. Med. Chem. vol. 36, pp. 1380-1386, 1993). The methylxanthine derivatives have been demonstrated as having tracheal relaxation and reduced heart rate functions. Some synthetic xanthine derivatives have shown affinity and selected effects on adenosine A₁ and A₂ receptor.

The compounds of invention has been proven to have favorable smooth muscle relaxation by the following pharmaceutical properties: activating adenosine acceptor, stimulating guanylyl cyclase of smooth muscles, inhibiting PDE (phosphodiesterases) activity, enhancing c-GMP in cell, and opening K⁺ channels. Base on above description it has been found that compounds 4, 9, 10, 12, 17, 18 could relax rabbit corpus cavernosal smooth muscle and increase intracavernous pressure (ΔICP). As shown in Table 4 the peak increased the intracavernous pressure (ΔICP) and duration of tumescence response to the compounds at 2 mg/kg in rabbits.

Table 2 show the rabbit corpus cavernosal smooth muscle relaxation induced by the compounds of this invention as shown by the experiments. The relaxation was not barely caused by NO (nitric oxide), less development of tolerance, and without seriously affecting the compensatory refluxed system.

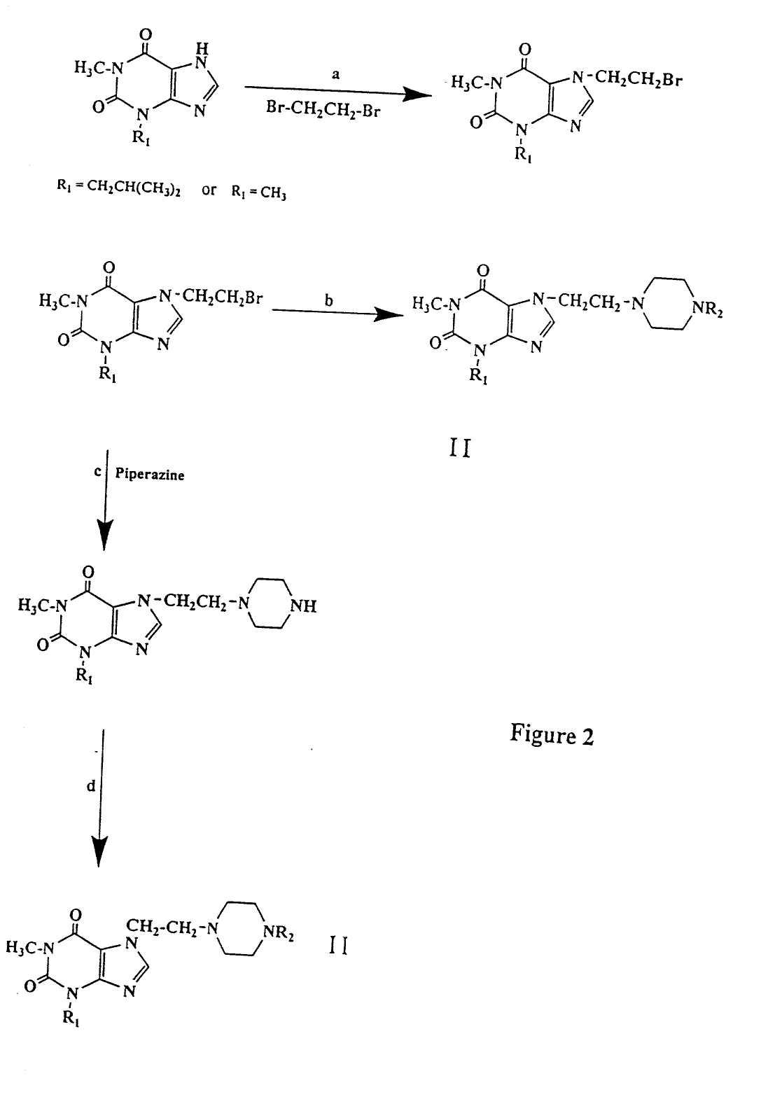

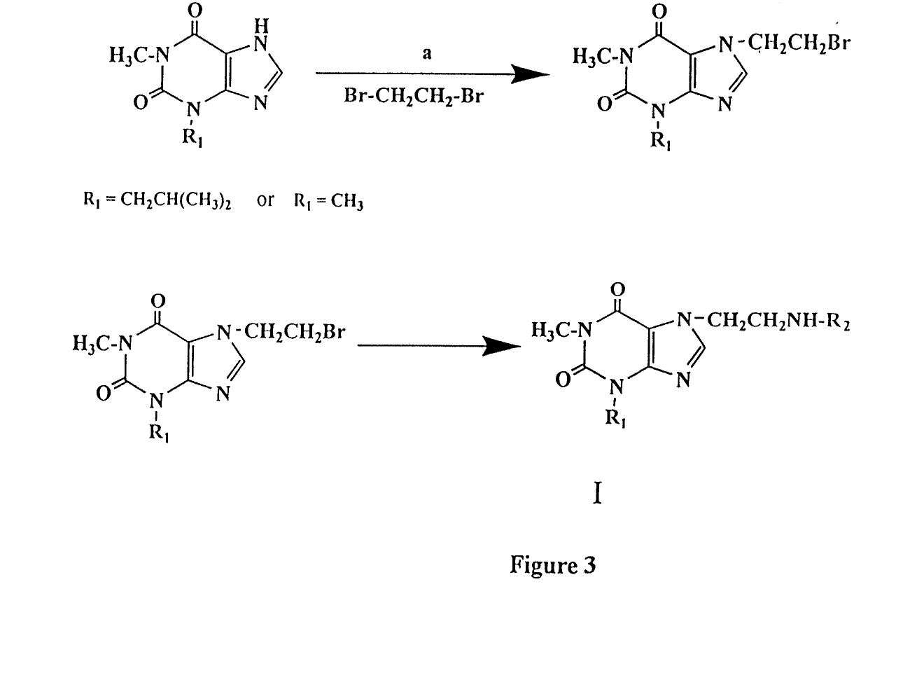

The method of synthesis of theophylline derivatives is shown in FIGS. 2 and 3, which show formulas I and II as the main structure. For the novel compounds above, different substitution on the two bases led to the change of their intermediate products, so that different processes have been developed. The process for compounds of formula II comprises the following step: 1) dissolving 3-isobutyl-1-methylxanthine (IBMX) into a halogen substituted ethylamine solution, for example 2- bromoethylamine solution; 2) stirring at mantle heater untill solid completely melts; 3) then adding NaOH to react at 150° C. overnight; 4) concentrating under reduced pressure to obtain white coarse crystal; 5) recrystalizing the product from methanol to obtain the pure white crystal compound D, N7-bromoethylamine3-isobutyl-1-methylxanthine.

### Preparation of Compounds 39-43 and 44

Into a three neck round bottom flask equipped with a mechanical stirrer, a thermometer, and a reflux condenser was placed chlorosulfonic acid and para-hydroxyl sulfonic acid sodium salt was added. The mixture was then heated at 65-67° C. and then poured onto crushed ice. The precipitate was separated by filteration. After washing with cold water, the product was dried under the reduced pressure. Methanol was added to dissolve the precipitate, then methylpiperazine was added to obtain a precipitate which was dissolved in methanol and then formalin and compound D (N7-bromoethylamine 3-isobutyl-1-methyl-xanthine) was added. The mixture was heated at 75° C. overnight, purified by column chromatography and eluated with a solvent system containing ethyl acetate and methanol, then it recrystallized from methanol to obtain compound 39. Compound 39 was dissolved in methanol, NaOH and ethyl bromide were added, heated at 75° C. and concentrated under reduced pressure, dissolved and recrystallized in methanol to obtain compound 40.

According the same method compound 41 was obtained by replacing ethyl bromide with propyl bromide. Compound 42, 43 or 44 were also obtained when replacing theophylline with IBMX, separately.

Para-hydroxybenzoic acid ethyl ester dissolved in methanol, formalin and acetic acid were added to react overnight. Then NH₃(aq) was added to obtain para-hydroxybenzoic amide. Formalin, acetic acid and N7-bromoethylaminute were added to obtain 3-isobutyl-1-methylxanthine to carry out a Mannich reaction to obtain compound 33. The product was purified from methanol, then NaOH was added to react with ethyl bromide to gobtain compound 34. By replacing ethyl bromide with propyl bromide, compound 35 was obtained. Compounds 36, 37 and 38 were also obtained by replacing theophylline with IBMX.

The process of preparation of the compounds of formula I show in FIG. 3 comprises the following step: 1) dissolving 3-isobutyl-1-methylxanthine (IBMX) in methanol and stirring with 2-bromoethylamine at mantle heater ; 2) reacting with NaOH ; and 3) recrystallizing with methanol to obtain a white crystal compound A (N7-bromoethyl 3-isobutyl-1-methylxanthine).

### Preparation of Compounds 1-8, 11-18, and 21-26

The process of preparation of the compounds 1-8 have different substituents. The process requires the following steps: 1) reflexing compound A with methanol; 2) then adding 1-Phenylpiperazine, 1-(2-pyridyl)piperazine, 1-(Pyrimidyl)piperazine, 1-(o-Metyhoxyphenyl)piperazine, 1-(2-Chlorophenyl)piperazine, 1-(m-Chlorophenyl)piperazine, N-benzylpiperazine, or 1-(4-Chlorophenyl)piperazine. Compounds 11˜18 were also obtained when theophylline base was replaced with IBMX.

As shown in FIG. 2, a solution of benzenesulfonyl chloride, piperazine, and methanol gave benzenesulfonyl piperazine. This product was dissolved in methanol and reacted with compound A (N 7-bromoethyl 3-isobutyl-1-methylxanthine) to obtain compound 21. When ethyl bromide was replaced with propyl bromide, compounds 22 and 23, were obtained when p-toluene-sulfonyl chloride or o-toluenesulfonyl chloride was replaced by benzenesulfonyl chloride, respectively. Compound 24, 25 or 26 were also obtained when theophylline base was replaced with IBMX, respectively.

### Prepapation of Compounds 9, 10, 45 and 46

Theophylline was dissolved methanol, 1,2-dibromoethane in, NaOH was added, heated under reflux, concentrated under reduced pressure, purified through silica gel column chromatography to obtain compound A. Compound A was dissolved in methanol and piperazine was added and reflexed to obtain compound B. Compound B was dissolved in methanol and 2-Furoyl Chloride or 4-chloronitrobenzene was added and reflexed to obtain compound 9 or 10.

4-chiorobenzene sulfonyl chloride and methylpiperazine in methanol were refluxed. N7- bromoethylamine 3-isobutyl-1-methylxanthine in methanol was added then refluxed to obtain compound 45. Compound 46 was obtained by replacing ethyl bromide and theophylline with IBMX.

After purification and crystallization, the products are individually tested for their physio-chemical properties, including elementary analysis MS, IR, ¹H-NMR (CDCl₃), and UV. The results are shown in Table 1. Appropriate experimental models are used to evaluate their pharmacological activities, thus ascertaining the compounds activity.

The compositions of this invention will include various excipients; carriers or diluents and pharmaceutically approved pH of processed salts in accordance to necessity to form composition with therapeutic efficacy. These pharmaceutical preparations may be in solid form for oral and rectal administration; liquid form or non-intestinal injection form; or ointment form for direct application on affected part. Such solid forms are manufactured according to common pharmaceutical preparation methods, which will include disintegrant like starch; sodium carboxymethylcellulose, adhesive like ethanol; glycerine, or magnesium stearic acid; lactose to obtain pharmaceutical preparation like tablets or filled into capsules of suppositories. Solution which include a compound of this ingredient could use buffers of phosphoric nature to adjust the pH to suitable level, before adding the adjutant; emulsifier to produce injection dose or other liquid preparation. In the present invention a compound or a pharmaceutical composition could be manufactured by mixing synthetic acid salts with various fundamental preparations to form ointments according to known pharmaceutical manufacturing methods. Pharmaceutical compositions manufactured according to this invention could be used on mammals to produce the efficacy of the main ingredient. General dosage could be adjusted according to the degree of symptoms, and normally a person will require 50 to 300 mg each time, three times per day.

### Pharmaceutical Activity

The pharmaceutical activity of the compounds of this invention have been proven by the following pharmaceutical experiments.

Compound 14 was dissolved in 10% absolute alcohol, 10% propylene glycol and 2% 1N HCl at 10 mM. Dilutions were made in distilled water. Glibenclamide was dissolved in 20% absolute alcohol and 80% DMSO (dimethyl sulfoxide ). Other drugs were dissolved in normal saline.

In vitro drugs

Compound 14 was dissolved in 10% absolute alcohol, 10% propylene glycol and 2% 1N HCl at 10 Mm-10⁻² M. Dilutions were made in redistilled water. 10⁻³ M glibenclamide was dissolved in 95% absolute alcohol, 10⁻¹ M dilutions were made in distilled water. 10⁻² M 1H-[1,2,4]Oxadiazolo[4,3-a]quinoxalin-1-one(ODQ) was dissolved in 100% DMSO (dimethyl sulfoxide), 10⁻³ M dilutions were made in absolute alcohol, 10⁻⁴ M dilutions were made in redistilled water. IBMX was dissolved in 10% DMSO,10⁻² M dilutions was made in redistilled water. 10⁻² M Levcromakalim was dissolved in 50% DMSO, dilutions were made in 50% redistilled water. 8-phenyltheopylline was dissolved in 80% absolute alcohol and 10⁻² M dilutions were made in 20% 0.2 M NaOH,while 10⁻³ M dilutions were made in redistilled water. 10⁻² M XAC (Xanthine amine congener) was dissolved in redistilled water and 10⁻³ M dilutions were made in redistilled water. 10⁻² M 3,7-dimethyl-1-propargyl xanthine (DMPX) was dissolved in 100% absolute alcohol, 10⁻³ M dilutions were made in redistilled water and 10⁻² M 8-(3-chlorostyryl) caffeine (CSC) was dissolved in DMSO,10⁻³ M dilutions were made in redistilled water. 10⁻² M alloxazine was dissolved in 100% absolute alcohol, 10⁻³ M dilutions were made in redistilled water. Other drugs were dissolved in distilled water.

The Krebs solution (mM) comprises NaCl 118, KCl 4.8, CaCl₂ 2.5, MgSO₄ 1.2, KH₂PO₄ 1.2, NaHCO₃ 24, and glucose 11.

Rabbit Corpus Cavernosal Strip Assay

Adult rabbit, weighing 200˜300 g were abdominally anaesthetized with pentobarbital sodium pentobarbital. The rabbit corpus cavernosal was remove immediately and placed in the Kreb solution equilibrated to a mixture of 95% O₂ and 5% CO₂ at room temperature (20˜25° C.). After the surrounding tissue was carefully removed, the trachea was cut into spiral shape with every turn having 5 mm segments, the two ends of the corpus cavernosal were clamped with frog-heart shaped clamps, one end was fixed at the bottom of 20 ml of a tissue bath made of physiological saline solution. The temperature was maintained at 37° C. The other end of the corpus cavernosal was connected to a force transducer and isometric contractions and beating rate of the right atria were recorded by COULBOURN AT-High-Speed Video Figure. After the samples were given 200 mg of contractions and reached equilibrium the following experiments were carried out:

(a) cumulative concentration-response curves

After the equilibrium was reached again for at least 60 minutes, different concentrations of the drug were used. For evaluation of the activity of rabbit corpus cavernosal, cumulative administration of drug 1×10⁻⁹˜1×10⁻⁴ M were carried out, and cumulative dose-response curve was obtained. The cumulative dose-response curve was the control group, The following experiments were performed:

(b) To examine the possible mechanisms of relaxation effects of corpus cavernosal, the aorta preparation of rats and rabbit was used.

(i) It was alos observed whether the relaxation effects of corpus cavernosal are affected by K⁺ channels.

Some of the K⁺ channels blocker eg. 10 mM tetraethylammonium chloride (TEA), 1 μ,M glibenclamide and 100 μM 4-aminopyridin (4-AP) were used prior to the addition of the compound. Cumulative administrations of the drug 1×10⁻⁸˜1×10⁻⁴ M were carried out again, and the inhibition of K⁺ channels blocker was obtained.

(ii) It was also observed whether the relaxation effects of corpus cavernosal are affected by cyclic GMP levels

Pretreatment with 100 μM N^{w}-nitro-L-arginine methyl ester (L-NAME), 10 μM methylene blue and 1 μM ODQ was carried out. Then cumulative applications of drug 1×10⁻⁹˜1×10⁻⁴ M were carried out. Inhibitions by NOS inhibitor were obtained.

(iii) It was also observed whether the relaxation effects of corpus cavernosal are affected by PDE( phosphodiesterase) inhibition.

1 μM phenylephrine solution was added to induce vasoconstriction and when the vasoconstriction reached stability, the product was repeatedly washed with Krebt's solution and 0.5 μM phenylephrine was added to cause contraction, again.

When the contraction reached the maximum, 1 μM IBMX (3-Isobutyl-1-Methylxanthine) was administered first, then 0.01 μM˜0.1 μM compound 14 was added, to determine whether corpus cavernosal relaxation of compound 14 are affected by IBMX.

Result

The mechanisms of corpus cavernosal relaxation

10 μM phenylephrine solution was added to induce vasoconstriction and when the vasoconstriction reached stability, cumulative administration of the drug 1×10⁻⁹˜1×10⁻⁴ M were carried out. Keeping relaxation cumulative dose-response curve as control group, the following experiments were performed:

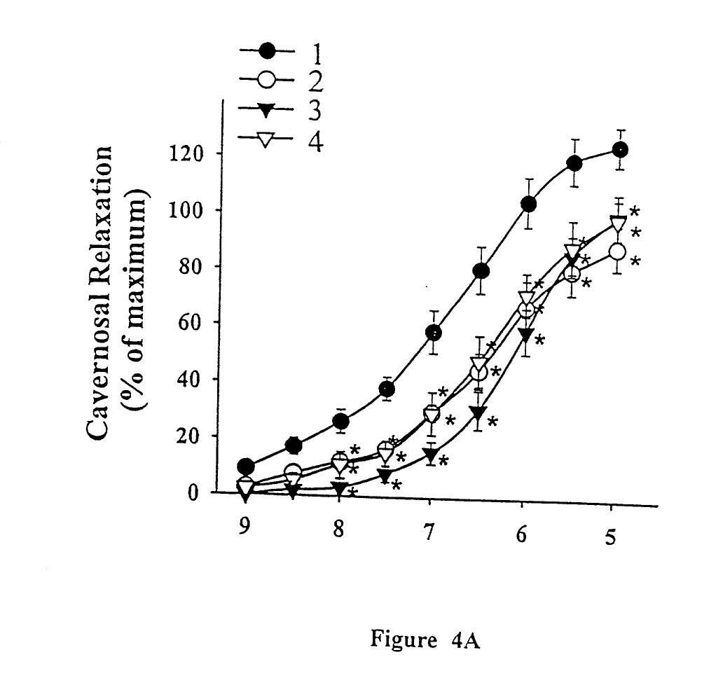

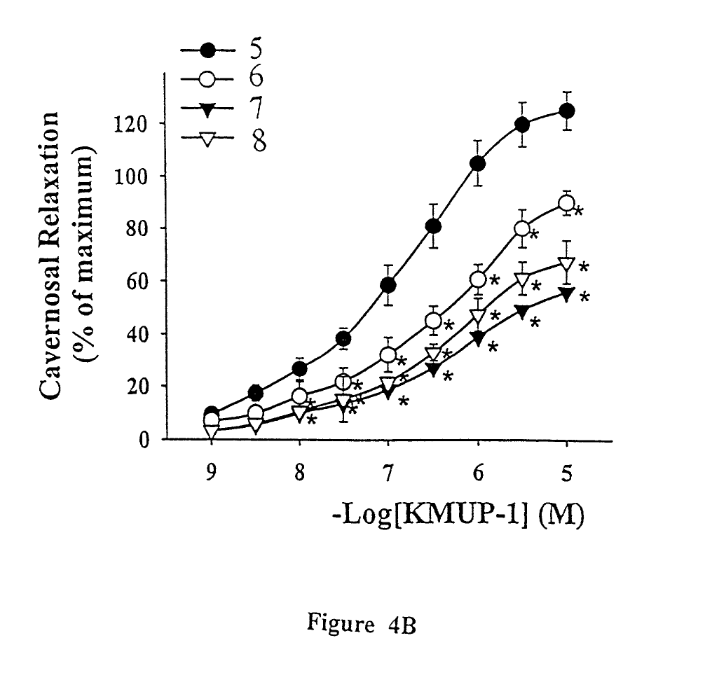

(i) FIG. 4 and Table 3 show the cumulative concentration-response curves to compound 14 against those K⁺ channels blocker, eg. TEA, glibenclamide and 4-AP pretreated the rabbit corpus cavernosal. This study shows that compound 14 has corpus cavernosal relaxation activator with K⁺ channels opening and cGMP breakdown inhibition activities.

(ii) The PDE (phosphodiesterase) inhibition activity affected the corpus cavernosal relaxation activity of compound 14

10 μM phenylephrine solution was added to induce vasoconstriction of endothelium-intact corpus cavernosal. When the vasoconstriction reached stability, the concentration-dependent vasorelaxations was produced by compound 14 (0.1, 0.5, and 1.00 μM). The relaxation cumulative dose-response curve was the control group. The endothelium-intact corpus cavernosal was repeatedly washed with Kreb's solution to move phenylephrine, untill after 60 minutes it reached stability, then the following experiments were performed:

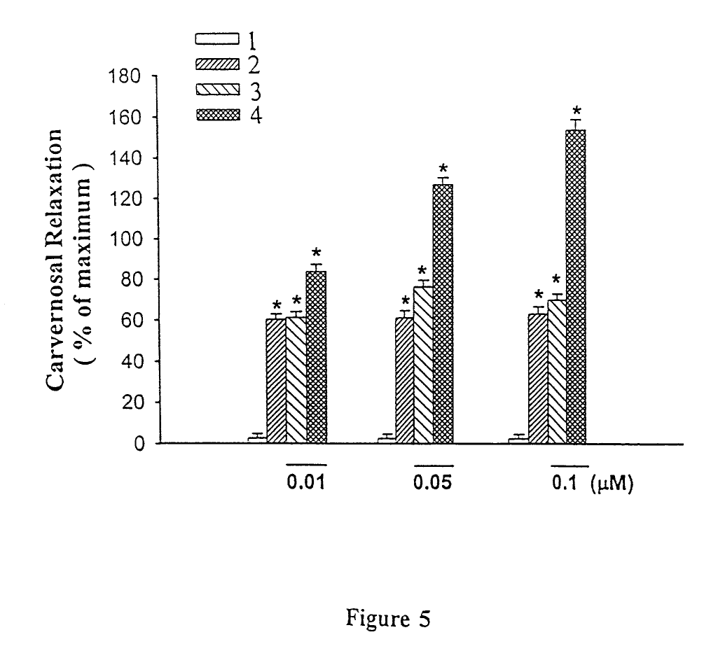

10 μM phenylephrine solution was added to the tissue bath to induce the vasoconstriction of endothelium-intact corpus cavernosal. When the vasoconstriction reached stability, the phenylephrine was repeatedly washed with Kreb's solution. Then 10/μM phenylephrine was administered. When the corpus cavernosal vasoconstriction reached stability IBMX (0.5 μM) was first administrate, then compound 14 (0.1, 0.5, and 1.0 μM) was added. FIG. 5 shows the relaxation cumulative concentration-response curves and IBMX has an additive effect of compound 14.

Phosphodiesterase five assay

Phosphodiesterase 5 (PDE₅) activity was determined as described by Seiler, S. et al. in which, [³H]cGMP is used as the substrate of the human platelets homogenates to determine the PDE activity, using Scintillation Proximity assay kit(SPA). Table 2 shows the inhibitory effect of phosphodiesterase in platelet.

Measurement of ICP

Male New Zealand white rabbits weighing 2-3 kg were used for measurement. After sedation with an intramuscular injection of ketamine 10 mg, the rabbits were anesthetized with intraperitoneal sodium 30/kg as needed. The animal breathed spontaneously. The rabbits were then placed in the supine position, and the body temperature was maintained at 37° C. using a heating pad and lamp. The femoral artery on one side was cannulated for monitoring of continuous systemic arterial pressure, the mean systemic arterial pressure and the heart rate via a Gould 23 ID pressure transducer were determined. Under sterile conditions, the skin overlying the penis was incised and the corpora cavernosa were exposed at the root of the penis.A25-guage needle was inserted into the corpus cavernosum for pressure recording (Gould, RS-3400). The needle was connected to a three-way stopcook, thus permitting the intracavernous injection of drugs. The tube was filled with heparizized saline (50IU/2-3 h) to prevent clotting. Table 4 shows the increase of intracarvernous pressure induced by the compounds of the invention.

### EXAMPLE 1

### Synthesis of Compound D

0.2 mole 3-isobutyl-1-methylxanthine (IBMX) was dissolved in 0.4 mole 2-bromoethylamine solution, and the solution was stirred at 100° C. mantle heater untill the solid completely melt. Then 125 ml 1.6 N NaOHl was added and the reaction was carried our for 3-5 hrs under 150° C. to complete the reaction. Then the product was concentrated under reduced pressure to obtain white coarse crystals which were recrystallized from methanol to obtain the pure white crystal compound D (N7-bromoethylamine 3-isobutyl-1-methylxanthine).

### EXAMPLE 2

### Synthesis of Compound 39˜41

One mole of para-hydroxyl sulfonic acid was reacted with 1 mole of chlorosulfonic acid for 30 minutes, then the liquid was poured into ice water. The precipitate of para-hydroxy sulfonyl chloride was collected and dried under reduced pressure.

With proper amount of methanol to dissolve the precipitate, 1 mole of methylpiperazine was added and reacted for 2 hrs. The product was dissolved in the 4 mole 30% formalin, then same mole amount of compound D (N7-bromoethylamine 3-isobutyl-1-methyl-xanthine) was added. Then 1% acetic acid -methanol solution was added to obtain compound 39 after 24 hours reaction. The product was purified through a silica gel column and was dissolved in methanol, 4% NaOH was added and 1 mole ethyl bromide was added to obtain compound 40. By replacing ethyl bromide with propyl bromide compound 41 was obtained.

### EXAMPLE 3

### Synthesis of Compound 42˜44

By the process of Example 2 compounds 42, 43 or 44 were obtain when theophylline was replaced with IBMX, respectively.

### EXAMPLE 4

### Synthesis of Compound 33˜35

Para-hydroxybenzoic acid was reacted with ethanol to obtain para-hydroxybenzoic acid ethyl ester by mediated with SOCl₂. The product was added to 33% NH₃(aq) , refluxed for 1 hour and recrystallized from methanol to obtain para-hydroxybenzoic amide. Formalin 4 moles times of para-hydroxybenzoic-amine solution was added in methanol, then the Mannich reaction was carried out with N7-bromoethylaminute 3-isobutyl-1-methylxanthine and trace of acetic acid. The product of compound 33 was obtained which was purified by silica gel chromatography.

The column was eluated with ethyl and methanol, the solution was concentrated under reduced pressure, recrystallized from methanol to obtain compound 33. Compound 33(1 mole) was dissolved in 100 ml methanol, reacted with 4% NaOH and 1 mole ethyl bromide, to obtain compound 34. Accordingly, compound 35 was obtain by replace ethyl bromide with propyl bromide.

### EXAMPLE 5

### Synthesis of Compound 36˜38

Follow the process described for Example 4 and replacing theophylline all with IBMX, separately, compounds 36, 37and 38 were obtained.

### EXAMPLE 6

### Synthesis of Compound A

0.2 mole of 3-isobutyl-1-methylxanthine (IBMX) was dissolved in methanol and mix with 0.4 mole 2-bromoethylamine solution. Then the solution was stirred at 100° C. in a mantle heater untill the solid was completely melt. Then 125 ml 1.6 N NaOH was added to react for 3-5 hours at 150° C. The reaction mixture was concentrated under reduced pressure to obtain white coarse crystals, which were recrystallized from methanol to obtain the white crystal compound A (N7-bromoethyl 3-isobutyl-1-methylxanthine).

### EXAMPLE 7

### Synthesis of Compound 21

Two moles benzenesulfonyl chloride and 2 moles of piperazine were dissolved in methanol and refluxed for 1 hour. The solution was concentrated under reduced pressure, recrystallized from methanol to obtain benzenesulfonyl piperazine. One mole of the product was dissolved in methanol, 1 mole of compound A (N7-bromoethyl 3-isobutyl-1-methylxanthine) was added and reflexed for 8 hours. The solution was concentrated under reduced pressure, purified by a silica gel column chromatography, eluated by methanol and ethyl acetate, concentrated under reduced pressure, recrystallized from methanol to obtain compound 21.

### EXAMPLE 8

### Synthesis of Compound 22˜23

Following the process of Example 7 and replacing p-toluene-sulfonyl chloride or o-toluenesulfonyl chloride with benzenesulfonyl chloride compounds 22 and 23 were obtained.

### EXMPLE 9

### Synthesis of Compound 24˜26

Following the process of Example 6 and 7 and replacing theophylline with IBMX compounds 24, 25 and 26 were obtained.

### EXAMPLE 10

### Synthesis of Compound 9 and 10

one of mole theophylline was dissolved in methanol and 3 moles of 1,2-dibromoethane were added with 2 mole NaOH to neutralization and the mixture was refluxed 5 hours. It was then concentrated under reduced pressure and purified by chromatography, eluated with methanol and ethyl acetate, then concentrated under reduced pressure to obtain compound A . Compound A was dissolved in methanol, 0.8 mole piperazine was added, the mixture refluxed and then concentrated under reduced pressure. The product B was was obtained.

Compound B was dissolved in methanol, then 2-Furoyl Chloride or 4-chloronitrobenzene were added, to reflux reaction, then concentrated under reduced pressure, which were recrystallization from methanol to obtain compound 9 or 10.

### EXAMPLE 11

### Synthesis of Compound 1˜8

Compound A was dissolved in methanol. Then 1-Phenylpiperazine, 1-(2-Pyrimidyl)piperazine, 1-(2-Pyridyl)piperazine, N-Benzylpiperazine, 1-(2-Chlorophenyl)piperazine, 1-(o-Methoxyphenyl)piperazine, 1-(m-Chlorophenyl)piperazine, or 1-(4-Chlorophenyl)piperazine was added, to reflux reaction to obtain compounds 1-8.

### EXAMPLE 12

### Synthesis of Compound 11˜18

By following the process of Example 11 and replacing theophylline with IBMX, compounds 11˜18 were obtained.

### EXAMPLE 13

### Synthesis of Compound 45

Equal mole of 4-chlorobenzene sulfonyl chloride and methylpiperazine were added in methanol and refluxed for 5 hours. This product and N7-bro moethylamine 3-isobutyl-1-methyixanthine were dissolved, in methanol and refluxed for 1 hour to obtain compound 45.

### EXAMPLE 14

### Synthesis Compound 46

By follow the process of Example 13, replacing theophylline with IBMX, the compound 46 was obtained.

TABLE 1
The physicochemical Data of N7-substituted xanthines

| | |
|---|---|
| MS(Scan | Compound |
| FAB+) | ¹H-NMR(CDCl₃) |

Compound 4

| | | | |
|---|---|---|---|
| 444.88 | δ: | 0.94-0.98 (d, 6H, 2 × CH₃), | 2.24-2.38 (m, 1H, CH), |
| | | 2.70 (t, 4H, 2 × CH₂), | 2.85 (t, 2H, NCH₂), |
| | | 3.04 (t, 4H, 2 × CH₂), | 3.42 (s, 3H, NCH₃), |
| | | 3.93-3.96 (d, 2H, CH₂), | 4.45 (t, 2H, NCH₂), |
| | | 6.97-7.01 (m, 2H, 2 × Ar—H), | 7.27-7.36 (m, 2H, 2 × |
| | | 7.69 (s, 1H, imidazole-H) | Ar—H), |

Compound 14

| | | | |
|---|---|---|---|
| 402.88 | δ: | 2.70 (t, 4H, 2 × CH₂), | 2.85 (t, 2H, NCH₂), |
| | | 3.04 (t, 4H, 2 × CH₂), | 3.42 (s, 3H, NCH₃), |
| | | 3.60 (s, 3H, NCH₃), | 4.45 (t, 2H, NCH₂), |
| | | 6.97-7.01 (m, 2H, 2 × Ar—H), | 7.27-7.36 (m, 2H, 2 × |
| | | 7.69 (s, 1H, imidazole-H) | Ar—H), |

Compound 17

| | | | |
|---|---|---|---|
| 398.46 | δ: | 2.75 (t, 4H, 2 × CH₂), | 2.89 (t, 2H, NCH₂), |
| | | 3.09 (t, 4H, 2 × CH₂), | 3.42 (s, 3H, NCH₃), |
| | | 3.61 (s, 3H, NCH₃), | 3.86 (s, 3H, OCH₃), |
| | | 4.49 (t, 2H, NCH₂), | 6.88-7.06 (m, 4H, 4 × |
| | | 7.72 (s, 1H, imidazole-H) | Ar—H), |

Compound 22

| | | | |
|---|---|---|---|
| 488.38 | δ: | 0.94-0.98 (d, 6H, 2 × CH₃), | 1.98 (m, 3H, Ar—CH₃) |
| | | 2.24-2.38 (m, 1H, CH), | 2.70 (t, 4H, 2 × CH₂), |
| | | 2.85 (t, 2H, NCH₂), | 3.04 (t, 4H, 2 × CH₂), |
| | | 3.42 (s, 3H, NCH₃), | 3.93-3.96 (d, 2H, CH₂), |
| | | 4.45 (t, 2H, NCH₂), | 6.97-7.01 (m, 2H, 2 × |
| | | 7.27-7.36 (m, 2H, 2 × Ar—H), | Ar—H), |
| | | | 7.69 (s, 1H, imidazole- |
| | | | H), |

Compound 25

| | | | |
|---|---|---|---|
| 446.38 | δ: | 1.98 (m, 3H, Ar—CH₃) | 2.70 (t, 4H, 2 × CH₂), |
| | | 2.85 (t, 2H, NCH₂), | 3.04 (t, 4H, 2 × CH₂), |
| | | 3.42 (s, 3H, NCH₃), | 3.60 (s, 3H, NCH₃), |
| | | 4.45 (t, 2H, NCH₂), | 6.97-7.01(m, 2H, 2 × |
| | | 7.27-7.36 (m, 2H, 2 × Ar—H), | Ar—H), |
| | | | 7.69 (s, 1H, imidazole- |
| | | | H), |

Compound 34

| | | | |
|---|---|---|---|
| 392.14 | δ | 0.94-0.98 (d, 6H, 2 × CH₃), | 1.4 (t, 2H, CH₂), |
| | | 2.24-2.38 (m, 1H, CH) | 2.28 (s, 2H, CH₂), |
| | | 2.85 (t, 2H, NCH₂) | 3.42 (s, 3H, NCH₃) |
| | | 3.93-3.96 (d, 2H, CH₂), | 4.13 (t, 3H, CH₃) |
| | | 4.45 (t, 2H, NCH₂), | 6.97-7.01 (m, 2H, 2 × |
| | | 7.27-7.36 (m, 1H, Ar—H), | Ar—H) |
| | | 7.98 (b, 1H, NH), | 7.69 (s, 1H, imidazole- |
| | | | H), |
| | | | 8.2 (b, 2H, NH₂) |

Compound 38

| | | | |
|---|---|---|---|
| 364.14 | δ: | 1.4 (t, 2H, CH₂), | 2.2 (t, 2H, CH₂), |
| | | 2.28 (s, 2H, CH₂), | 2.85 (t, 2H, NCH₂), |
| | | 3.42 (s, 3H, NCH₃), | 3.60 (s, 3H, NCH₃), |
| | | 4.13 (t, 3H, CH₃), | 4.45 (t, 2H, NCH₂), |
| | | 6.97-7.01 (m, 2H, 2 × Ar—H), | 7.27-7.36 (m, 1H, Ar— |
| | | 7.69 (s, 1H, imidazole-H) | H), |
| | | 8.2 (b, 2H, NH₂) | 7.98 (b, 1H, NH) |

Compound 40

| | | | |
|---|---|---|---|
| 511.14 | δ: | 0.94-0.98 (d, 6H, 2 × CH₃), | 1.4 (t, 2H, CH₂), |
| | | 1.98 (m, 3H, Ar—CH₃), | 2.24-2.38 (m, 1H, CH), |
| | | 2.28 (s, 2H, CH₂), | 2.70 (t, 4H, 2 × CH₂), |
| | | 2.85 (t, 2H, NCH₂), | 3.04 (t, 4H, 2 × CH₂), |
| | | 3.42 (s, 3H, NCH₃), | 3.93-3.96 (d, 2H, CH₂), |
| | | 4.13 (t, 3H, CH₃), | 4.45 (t, 2H, NCH₂), |
| | | 6.97-7.01 (m, 2H, 2 × Ar—H), | 7.27-7.36 (m, 1H, Ar— |
| | | 7.69 (s, 1H, imidazole-H), | H), |
| | | | 7.98 (b, 1H, NH) |

Compound 44

| | | | |
|---|---|---|---|
| 483.14 | δ: | 1.4 (t, 2H, CH₂), | 1.8 (t, 2H, CH₂), |
| | | 1.98 (m, 3H, Ar—CH₃), | 2.28 (s, 2H, CH₂), |
| | | 2.70 (t, 4H, 2 × H₂), | 2.85 (t, 2H, NCH₂), |
| | | 3.04 (t, 4H, 2 × CH₂), | 3.42 (s, 3H, NCH₃), |
| | | 3.60 (s, 3H, NCH₃), | 4.13 (t, 3H, CH₃) |
| | | 4.45 (t, 2H, NCH₂), | 6.97-7.01 (m, 2H, 2 × |
| | | 7.27-7.36 (m, 1H, Ar—H), | Ar—H), |
| | | 7.98 (b, 1H, NH), | 7.69 (s, 1H, imidazole- |
| | | | H) |

Compound 45

| | | | |
|---|---|---|---|
| 436.14 | δ: | 0.94-0.98 (d, 6H, 2 × CH₃), | 1.98 (m, 3H, Ar—CH₃), |
| | | 2.24-2.38 (m, 1H, CH), | 2.28 (s, 2H, CH₂), |
| | | 2.70 (t, 4H, 2 × CH₂), | 2.85 (t, 2H, NCH₂), |
| | | 3.04 (t, 4H, 2 × CH₂), | 3.42 (s, 3H, NCH₃), |
| | | 3.93-3.96 (d, 2H, CH₂), | 4.13 (t, 3H, CH₃), |
| | | 4.45 (t, 2H, NCH₂), | 6.97-7.01 (m, 2H, 2 × |
| | | 7.27-7.36 (m, 1H, Ar—H), | Ar—H), |
| | | 7.98 (b, 1H, NH), | 7.69 (s, 1H, imidazole- |
| | | | H), |

TABLE 2
Rabbit Corpus Cavernosal Relaxation IC₅₀

| | PDE₅ | Rabbit Corpus Cavernosal |
|---|---|---|
| compound | (human platelet) IC₅₀(nM) | Relaxation IC₅₀ |
| 4 | 3.9 ± 0.1 | 7.16 ± 0.09 |
| 7 | 4.2 ± 0.2 | 7.13 ± 0.06 |
| 14 | 3.8 ± 0.2 | 7.84 ± 0.08 |
| 17 | 6.2 ± 0.2 | 7.64 ± 0.07 |
| 23 | 5.2 ± 0.1 | 7.38 ± 0.04 |
| 26 | 4.8 ± 0.2 | 7.42 ± 0.09 |
| 34 | 0.4 ± 0.2 | 8.13 ± 0.05 |
| 37 | 0.3 ± 0.1 | 8.03 ± 0.04 |
| 35 | 0.4 ± 0.1 | 8.25 ± 0.06 |
| 38 | 0.6 ± 0.2 | 8.16 ± 0.07 |
| 39 | 0.6 ± 0.2 | 8.16 ± 0.07 |
| 42 | 0.6 ± 0.2 | 8.16 ± 0.07 |
| 40 | 0.6 ± 0.1 | 8.27 ± 0.04 |
| 43 | 0.7 ± 0.2 | 8.15 ± 0.06 |
| 41 | 0.8 ± 0.2 | 8.30 ± 0.07 |
| 44 | 0.9 ± 0.1 | 8.25 ± 0.08 |
| 45 | 0.6 ± 0.2 | 7.92 ± 0.07 |
| 46 | 5.2 ± 0.1 | 7.96 ± 0.03 |

TABLE 3
Rabbit Corpus Cavernosal Relaxation IC₅₀ on K⁺ channels blocker

| Drug pretreatment | Dose | −Log EC₅₀ |
|---|---|---|
| Control | | 7.19 ± 0.09 |
| TEA | 10 mM | 5.037 ± 0.05 |
| Glibenclamide | 1 μM | 6.57 ± 0.15 |
| 4-AP | 100 μM | 5.83 ± 0.17 |
| L-NAME | 100 μM | 6.51 ± 0.08 |
| Methylene blue | 10 μM | 6.51 ± 0.06 |
| ODQ | 1 μM | 6.79 ± 0.12 |

TABLE 4
Peak increased intracavernous pressure (ΔICP) and duration of tumescence
response to compounds at 2 mg/kg in rabbits

| compound | ΔICP (mmHg) | Duration (min) |
|---|---|---|
| Compound 10 | 12 ± 1.6 | 13 ± 2.1 |
| Compound 4 | 14 ± 2.1 | 14 ± 1.1 |
| Compound 9 | 25 ± 1.3 | 16 ± 1.2 |
| Compound 12 | 23 ± 1.5 | 15 ± 1.3 |
| Compound 18 | 26 ± 1.4 | 18 ± 1.3 |

What is claimed is:

1. A compound containing the theophylline moiety of formula I, 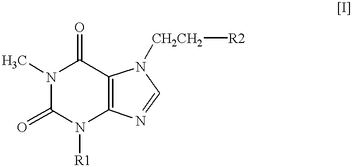
wherein
R¹ is —(CH₂)ₙCH₃; R₂ is a member selected from the group 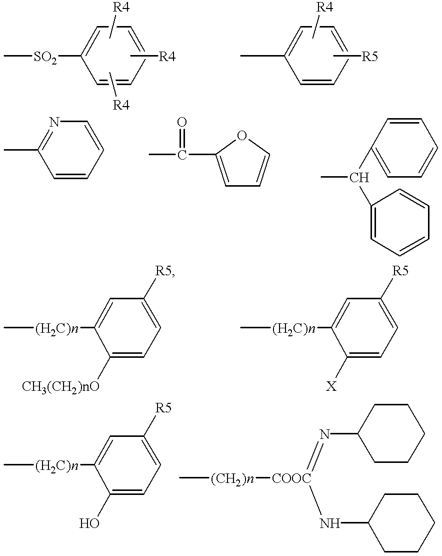
wherein
R₄ is a member selected from the group of H,—(CH₂)ₙCH₃, X, —NH₂ and —NO₂;
R₅ is a member selected from the group of H, 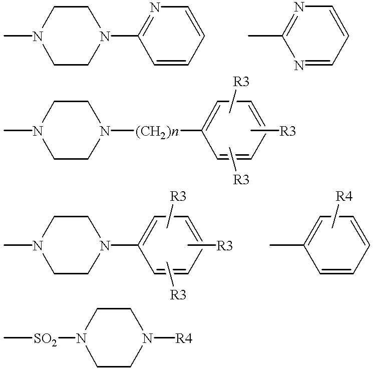
wherein
R₃ is a member selected from the group of halogen, hydroxyl group saturated 1-3 straight chain carbon or has a substitution group of one hydrogen; n is 0, 1, 2 or 3.

2. A compound containing the theophylline moiety of formula II, 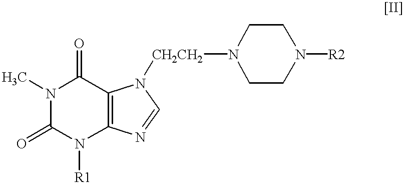
wherein
R₁ is —(CH₂)ₙCH₃ ; R₂ is a member selected from the group of 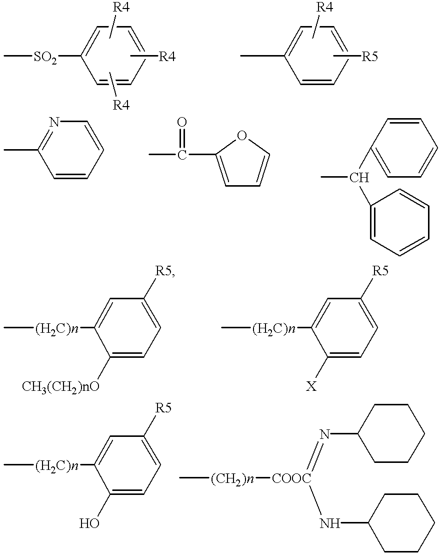
wherein
R₄ is a member selected from the group of H, —(CH₂)ₙCH₃, X, —NH₂ and —NO₂;
R₅ is a member selected from the group of H, 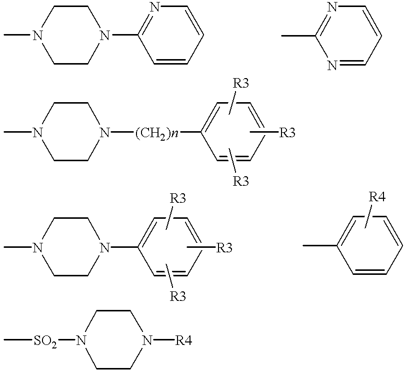
R₃ is a member selected from the group of halogen, hydroxyl group (OH), saturated 1-3 straight chain carbon or group substituting one hydrogen; n is 0, 1, 2 or 3.

3. The process of preparation of a compound of formula as defined in claim 1 which comprises the steps of 1) dissolving 3-isobutyl-1-methylxanthine (IBMX) in methanol and stirring with 2-bromoethylamine at mantle heater; 2) reacting with NaOH; and 3) recrystallizing from methanol to obtain a white crystal compound A(N-7-bromoethyl 3-isobutyl-1-methylxanthine).

4. The process of preparation of a compound of formula II as defined in claim 2 which comprises the 1) dissolving 3-isobutyl-1-methylxanthine (IBMX) into a halogen substituted ethylamine solution, 2) stirring at mantle heater until the solid completely melts; 3) then adding NaOH to react at 150° C. overnight; 4) concentrating under reduced pressure to obtain white coarse crystal; 5) recrystalizing the product from methanol to obtain the pure white crystal compound D, N7-bromoethylamine 3-isobutyl-1-methylxanthine.

5. A pharmaceutical composition which has corpus cavernosal relaxation activity containing a compound of formula I as defined in claim 1.

6. A pharmaceutical composition which has corpus cavernosal relaxation activity containing a compound of formula II as defined in claim 2.

7. The composition according to claim 5 which contains diluents and excipients.

8. The composition according to claim 6 which contains diluents and excipients.
